(19) United States
(12) Patent Application Publication
(10) Pub. No.: US 2026/0182876 A1
(43) Pub. Date: Jul. 2, 2026

(54) SIGNAL PROCESSING APPARATUS AND SIGNAL PROCESSING METHOD

(71) Applicant: SONY GROUP CORPORATION, TOKYO (JP)

(72) Inventors: YASUHIDE HYODO, TOKYO (JP); KIYOSHI YOSHIKAWA, TOKYO (JP)

(21) Appl. No.: 19/133,818

(22) PCT Filed: Nov. 22, 2023
(86) PCT No.: PCT/JP2023/042048

§ 371 Date: May 29, 2025

(30) Foreign Application Priority Data
Dec. 5, 2022 (JP) 2022-194285

(51) Int. Cl. A61B 5/16; A61B 5/00; A61B 5/11; G06F 18/15

(52) U.S. Cl. CPC A61B 5/165; A61B 5/1118; A61B 5/7203; A61B 5/7221; A61B 5/7267; A61B 5/7278; G06F 18/15

(57) ABSTRACT

The present technology relates to a signal processing apparatus and a signal processing method that make it possible to improve the accuracy in estimating an emotion. A signal processing apparatus extracts an input feature amount on the basis of a measured biological signal, corrects a normalization coefficient according to a context related to a user, normalizes the input feature amount using the corrected normalization coefficient, and outputs a prediction label of an emotion state correspondingly to the normalized input feature amount using a machine learning model created in advance. The present technology can be applied to an emotion estimation processing apparatus.

### TECHNICAL FIELD

The present technology relates to a signal processing apparatus and a signal processing method, and, in particular, to a signal processing apparatus and a signal processing method that make it possible to improve the accuracy in estimating an emotion.

### BACKGROUND ART

An emotion is a kind of feeling, and corresponds to a state of a temporary feeling that is caused suddenly and that disappears in a short period of time, where the temporary feeling exhibits a great response amplitude. When there is a change in an emotion of a person, physiological responses such as brain waves, heartbeat, and sweating appear on a body surface. An emotion estimation system that estimates an emotion of a person reads these physiological responses in the form of a biological signal using a sensor device, extracts, using signal processing, a feature amount such as a physiological index that contributes toward emotions, and estimates an emotion of a user from the feature amount using a model obtained by machine learning. The types of emotions are classified using two bases that are a comfortable or uncomfortable feeling and a degree of arousal.

However, when a physiologically adequate emotion estimation model is created in a laboratory environment and the created model is applied to an actual environment, contextual dependencies have an impact thereon, where the contextual dependency means that physiological responses due to an emotion of a person are dependent on the context (any state of the person) (refer to Non-Patent Literature 1).

On the other hand, a response range (a reference range) for a state of an emotion of a person differs depending on an application, where the response range for the emotion state is a range in which an application responds to (detects) the emotion state. Here, the response range for an emotion state is described using high and low degrees of arousal (being stressed and being relaxed) as an example (refer to Patent Literature 1).

In the case of, for example, an emotion meditation application, a person is essentially relaxed, and it is necessary for the application to visualize slight nuances of a degree of arousal in a state of being relaxed. On the other hand, there is a need to visualize both an arousal state and a state of being relaxed within a day or in a long period of time with respect to a lifelog of activities (visualization of a stressful state in an everyday life).

### CITATION LIST

### Non-Patent Literature

Non-Patent Literature 1: Bamert M and Inauen J (2022) Physiological stress reactivity and recovery: Some laboratory results transfer to daily life. Front. Psychol. 13:943065. doi: 10.3389/fpsyg.2022.943065, Internet search <https://www.frontiersin.org/articles/10.3389/fpsyg.2022.943065/full, searched on Dec. 5, 2022>

### Patent Literature

Patent Literature 1: Japanese Patent Application Laid-open No. 2016-106689

### DISCLOSURE OF INVENTION

### Technical Problem

It is necessary for an emotion estimation system to control a sensitivity change caused due to the behavioral context (behavior state) for a physiological response and disclosed in Non-Patent Literature 1 indicated above, in order to get a response range for a degree of arousal according to an application to estimate an emotion accurately, as described above.

The present technology has been made in view of the circumstances described above, and it is an object of the present technology to make it possible to improve the accuracy in estimating an emotion.

### Solution to Problem

A signal processing apparatus according to an aspect of the present technology includes a feature amount extracting section that extracts an input feature amount on the basis of a measured biological signal; a response range correcting section that corrects a normalization coefficient according to a context related to a user; a normalization section that normalizes the input feature amount using the normalization coefficient corrected by the response range correcting section; and a section for chronologically labeling emotion states, the chronologically labeling section outputting a prediction label of an emotion state correspondingly to the normalized input feature amount using a machine learning model created in advance.

In the aspect of the present technology, an input feature amount is extracted on the basis of a measured biological signal. Then, a normalization coefficient is corrected according to a context related to a user; the input feature amount is normalized using the corrected normalization coefficient; and a prediction label of an emotion state is output correspondingly to the normalized input feature amount using a machine learning model created in advance.

### BRIEF DESCRIPTION OF DRAWINGS

FIG. 1 is a block diagram of an example of a configuration of an emotion estimation processing apparatus according to an embodiment of the present technology.

FIG. 2 is a functional block diagram of examples of functional configurations of an APP standard acquiring section, a normalization section, a section for chronologically labeling emotion states, and a stabilization processing section that are illustrated in FIG. 1.

FIG. 3 illustrates a first example of correcting a response range according to an application standard.

FIG. 4 illustrates a second example of correcting a response range according to an application standard.

FIG. 5 illustrates a third example of correcting a response range according to an application standard.

FIG. 6 illustrates an example of processing of correcting a range according to a behavior state.

FIG. 7 is a flowchart used to describe emotion estimation processing performed by the emotion estimation processing apparatus illustrated in FIG. 1.

FIG. 8 is a flowchart used to describe response range adjusting processing of Step S13 in FIG. 7.

FIG. 9 is a block diagram of an example of a configuration of an emotion estimation processing apparatus according to a second embodiment of the present technology.

FIG. 10 is a flowchart used to describe emotion estimation processing performed by the emotion estimation processing apparatus illustrated in FIG. 9.

FIG. 11 illustrates an example of processing of correcting a range according to a location state.

FIG. 12 is a block diagram of an example of a configuration of a computer.

### MODE(S) FOR CARRYING OUT THE INVENTION

Embodiments for carrying out the present technology are described below. The description is made in the following order.
1. First Embodiment (Basic Configuration)
2. Second Embodiment (Addition of Signal Quality Determining Section)
3. Others

### 1. First Embodiment (Basic Configuration)

### <Example of Configuration of Emotion Estimation Processing Apparatus>

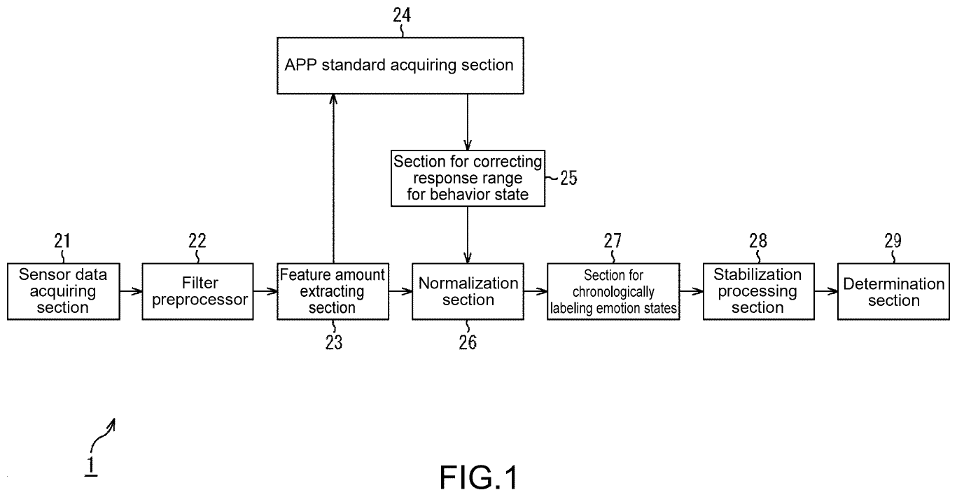

FIG. 1 is a block diagram of an example of a configuration of an emotion estimation processing apparatus according to a first embodiment of the present technology.

An emotion estimation processing apparatus 1 illustrated in FIG. 1 is a signal processing apparatus that detects a signal related to a state of a living body (hereinafter referred to as a biological signal) and that estimates a state of an emotion of the living body on the basis of the detected biological signal. For example, the emotion estimation processing apparatus 1 is directly attached to a living body in order to detect a biological signal. For example, an emotion-state-estimation-target living body (hereinafter referred to as a target living body) is a human. Note that a living body that is a target for the emotion estimation processing apparatus 1 is not limited to humans.

Specifically, when the emotion estimation processing apparatus 1 is canal headphones or a headband, an ear is a measurement part. When the emotion estimation processing apparatus 1 is virtual reality (VR) goggles, a forehead is a measurement part. When the emotion estimation processing apparatus 1 is a band, an arm or leg to which the band is attached is a measurement part.

Note that the emotion estimation processing apparatus 1 may be a server that receives a detected biological signal and that performs emotion estimation processing, where the server is separate from an apparatus that detects the biological signal.

In FIG. 1, the emotion estimation processing apparatus 1 includes a sensor data acquiring section 21, a filter preprocessor 22, a feature amount extracting section 23, an application (hereinafter referred to as an APP) standard acquiring section 24, a section 25 for correcting a response range for a behavior state, a normalization section 26, a section 27 for chronologically labeling emotion states, a stabilization processing section 28, and a determination section 29.

For example, the sensor data acquiring section 21 acquires a biological signal (raw data) from a sensor (not illustrated) included in the emotion estimation processing apparatus 1.

Note that, for example, the sensor may be a sensor that is brought into contact with a target living body, or may be a sensor that is not brought into contact with the target living body. For example, the sensor is a sensor that detects information (a biological signal) regarding at least one of a brain wave, sweating (mental), a pulse wave, an electrocardiogram, a blood flow, a continuous blood pressure, breathing, a skin temperature, a facial expression myoelectric potential, an electrooculogram, an eyeblink, or a specific component contained in saliva.

The filter preprocessor 22 performs preprocessing such as bandpass filtering or denoising on a biological signal acquired by the sensor data acquiring section 21. The filter preprocessor 22 outputs, to the feature amount extracting section 23, the biological signal on which preprocessing has been performed.

Using the biological signal supplied by the filter preprocessor 22, the feature amount extracting section 23 extracts a feature amount as a model input variable used to estimate an emotion state. The feature amount extracting section 23 outputs the extracted feature amount to the APP standard acquiring section 24 and the normalization section 26.

Note that the feature amount is not limited to an amount of a physiologically known feature. For example, the feature amount extracting section 23 may also perform signal processing performed to extract, in a data-driven manner, a feature amount that contributes toward emotions, using, for example, deep learning or autoencoder.

Here, the response range for an emotion state differs depending on an application, as described above.

The description is made using high and low degrees of arousal (being stressed and being relaxed) as an example. In the case of, for example, an emotion meditation application, a person is essentially relaxed, and it is necessary for the application to visualize slight nuances of a degree of arousal in a state of being relaxed. On the other hand, in the case of an application of a lifelog of activities (visualization of a stressful state in an everyday life), there is a need to visualize both an arousal state and a state of being relaxed within a day or in a long period of time. Note that the degree of arousal described above is used to describe an example of an emotion state.

The APP standard acquiring section 24 converts a normalization coefficient according to a response range for an emotion state for an application to adjust the response range, where the application is started in the emotion estimation processing apparatus 1.

In other words, a feature amount of data and a normalization coefficient for the feature amount are held in a memory (not illustrated) of the APP standard acquiring section 24 in advance, the feature amount being used upon creation of a reference model for each application. The reference model refers to a machine learning model used by the section 27 for chronologically labeling emotion states. This will be described in detail later.

The APP standard acquiring section 24 selects a reference model corresponding to an application to be started, and acquires a normalization coefficient for a feature amount for the selected reference model from the memory. The APP standard acquiring section 24 derives a conversion table used to convert the normalization coefficient according to a response range for an emotion state for the application. The APP standard acquiring section 24 converts the acquired normalization coefficient using the derived conversion table. The APP standard acquiring section 24 outputs the converted normalization coefficient to the section 25 for correcting a response range for a behavior state.

The section 25 for correcting a response range for a behavior state corrects the normalization coefficient in consideration of an impact of a state of a behavior of a user to correct the response range adjusted by the APP standard acquiring section 24.

In other words, the section 25 for correcting a response range for a behavior state performs, for example, gain adjustment according to a behavioral context obtained from an inertial measurement unit (IMU) with respect to the normalization coefficient converted by the APP standard acquiring section 24. Note that the behavioral context refers to, for example, a behavior state.

The section 25 for correcting a response range for a behavior state outputs, to the normalization section 26, the normalization coefficient on which gain adjustment has been performed.

The normalization section 26 normalizes the feature amount supplied by the feature amount extracting section 23, using the normalization coefficient supplied by the section 25 for correcting a response range for a behavior state. The normalization section 26 outputs the normalized feature amount to the section 27 for chronologically labeling emotion states.

The section 27 for chronologically labeling emotion states includes a plurality of reference models for respective applications. The section 27 for chronologically labeling emotion states uses, as input, chronological feature amounts, from among feature amounts supplied by the normalization section 26, that are in a sliding window. The section 27 for chronologically labeling emotion states performs identification on chronological prediction labels of emotion states using the reference models, and labels the emotion states using the prediction labels on which identification has been performed.

The section 27 for chronologically labeling emotion states outputs, to the stabilization processing section 28, chronological data of prediction labels that is a result of chronologically labeling the emotion states. Here, degrees of reliability of the prediction labels that are obtained using the reference models are also output.

In general, an identification model used to analyze chronological data or to perform natural language processing is assumed to be a reference model in an approach of chronologically labeling emotion states. Specific examples include a support vector machine (SVM), a k-nearest neighbor (k-NN), linear discriminant analysis (LDA), hidden Markov models (HMMs), conditional random fields (CRFs), a structured output support vector machine (SOSVM), a Bayesian network, a recurrent neural network (RNN), and a long short-term memory (LSTM). However, the approach is not limited.

Using the chronological data of the prediction labels of the emotion states that is supplied by the section 27 for chronologically labeling emotion states, the stabilization processing section 28 performs a weighted summation of the chronological prediction labels of the emotion states with the degrees of reliability of the prediction labels of the emotion states in a sliding window, and outputs a representative value of the prediction labels and a degree of reliability of the representative value of the prediction labels to the determination section 29 as an emotion estimation result. The degree of reliability of a representative value of prediction labels is a degree of reliability obtained when the representative value of prediction labels is calculated.

Specifically, the stabilization processing section 28 performs a weighted summation of the prediction labels of the emotion states in a sliding window with degrees of reliability of the prediction labels to calculate a degree of reliability of a representative value of the prediction labels in a sliding window. Further, the stabilization processing section 28 performs threshold processing on the degree of reliability of the representative value of the prediction labels, and outputs the representative value of the prediction labels as an emotion estimation result.

A degree of reliability r of a representative value of prediction labels is calculated using a formula (1) indicated below.

$$[ Math . 1 ]$$

$$r ( t ) = {\sum }_{i} {w}_{i} {c}_{i} {s}_{i} {y}_{i} Δ {t}_{i} / {\sum }_{i} {w}_{i} Δ {t}_{i} \qquad ( 1 )$$

Here, i represents an event number of an event of a plurality of events detected in a sliding window. y represents a prediction label of an emotion state, c represents a degree of reliability of the prediction label that is obtained using a reference model, and Δtᵢ represents a period of time for which an i-th event continues. Further, w represents a forgetting weight, and the weight is set smaller for a more previous time.

With respect to degrees of reliability of prediction labels of emotion states for a plurality of events detected in a sliding window, a degree of reliability of a representative value of prediction labels in the sliding window is calculated in the form of consecutive values of [−1 1] using the formula (1) described above.

Further, threshold processing is performed on a degree of reliability r that is output of the formula (1), and the degree of reliability is substituted for a formula (2) indicated below. Accordingly, a representative value z of prediction labels is calculated as an emotion estimation result.

$$[ Math . 2 ]$$

$$z ( t ) = \{ \begin{matrix}0 & ( if r ( t ) < 0 ) \\ 1 & ( if r ( t ) > 0 ) \\ previous value & ( otherwise )\end{matrix} \qquad ( 2 )$$

In the formula (2), a numerical value of the representative value z of prediction labels that is an emotion estimation result is dependent on a definition of the prediction label y of an emotion state of a user.

When, for example, a prediction label of an emotion state represents a result of identifying a degree of arousal, the prediction label of an emotion state is defined by two classes that correspond to 0 or 1, where 0 and 1 respectively represent low and high degrees of arousal. In this case, a state of an emotion of a user at a corresponding time is identified as a low degree of arousal (being relaxed) when a representative value z of prediction labels that is an emotion estimation result is 0. Further, the state of the emotion of the user at a corresponding time is identified as a high degree of arousal (an arousal state and a state of being concentrated) when the representative value z of prediction labels that is an emotion estimation result is 1.

The determination section 29 determines a state of an emotion of a target living body using a representative value of prediction labels and a degree of reliability of the representative value of prediction labels, where the representative value and the degree of reliability are supplied by the stabilization processing section 28.

### <Configurations of Respective Structural Elements>

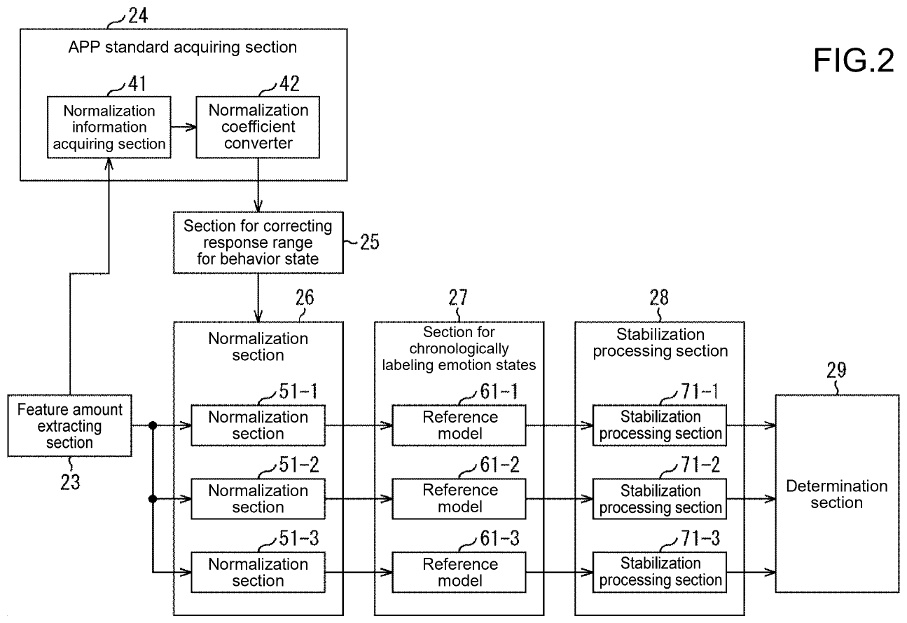

FIG. 2 is a functional block diagram of examples of functional configurations of the APP standard acquiring section 24, the normalization section 26, the section 27 for chronologically labeling emotion states, and the stabilization processing section 28 that are illustrated in FIG. 1.

Note that the section 27 for chronologically labeling emotion states that is illustrated in FIG. 2 includes a reference model created in advance for each application, as described above. A response range for an emotion state differs depending on an application. In other words, the section 27 for chronologically labeling emotion states includes a plurality of reference models (for example, three reference models 61-1 to 61-3) with different degrees of emotion states. For example, the reference model includes a regression model or an identification model.

For example, the description is made using high and low degrees of arousal as an example. The reference model 61-1 is a model for a state with a low degree of arousal (such as a degree-of-relaxing estimating model described later). The reference model 61-2 is a model for a state with a moderate degree of arousal. The reference model 61-3 is a model for a state with a high degree of arousal (such as a degree-of-arousal estimating model described later). The reference models 61-1 to 61-3 are referred to as reference models 61 when there is no particular need to distinguish between the reference models 61-1 to 61-3.

As described above, the emotion estimation processing apparatus 1 estimates a state of an emotion of a target living body using a plurality of reference models 61 of different response ranges for emotion states, the plurality of reference models 61 being provided to the section 27 for chronologically labeling emotion states.

Note that the target living body upon creation of the reference model 61 is normally different from a target living body of which an emotion is estimated by the emotion estimation processing apparatus 1.

In FIG. 2, the APP standard acquiring section 24 includes a normalization information acquiring section 41 and a normalization coefficient converter 42. The APP standard acquiring section 24 acquires an application trigger issued by an application started in the emotion estimation processing apparatus 1.

The normalization information acquiring section 41 holds a feature amount used upon creation of a reference model for each application or a normalization coefficient for the feature amount. Further, a feature amount is supplied to the normalization information acquiring section 41 by the feature amount extracting section 23.

The normalization information acquiring section 41 acquires, according to the type of application obtained from the application trigger, the held feature amount used upon creation of the reference model. Then, the normalization information acquiring section 41 outputs the acquired feature amount and the feature amount extracted by the feature amount extracting section 23 to the normalization coefficient converter 42. Note that, in the following description, the feature amount used upon creation of a reference model is referred to as a feature amount used upon creation and the feature amount extracted by the feature amount extracting section 23 is referred to as an input feature amount when there is a need to distinguish between the feature amounts.

Further, the normalization information acquiring section 41 acquires, according to the type of application obtained from the application trigger, the held normalization coefficient for the feature amount used upon creation of the reference model. Then, the normalization information acquiring section 41 outputs the acquired normalization coefficient to the normalization coefficient converter 42.

When, for example, a normalization method is min-max normalization (normalization is performed such that a maximum and a minimum of a distribution of feature amounts are respectively set to 0 and 1), X, which corresponds to a feature amount before normalization, is normalized using “(X−Xmin)/(Xmax−Xmin)”. In this case, the normalization information acquiring section 41 acquires Xmin and Xmax as normalization coefficients (Xmax represents a maximum of a feature amount, and Xmin represents a minimum of the feature amount). More generally, the normalization of a feature amount corresponds to space mapping. Thus, the normalization coefficient is represented using a mapping function such as g1( ).

Note that the normalization method is not limited to the min-max normalization, and another method such as z-score normalization (standardization is performed using an average and a variance of a distribution of feature amounts) may be adopted. When the normalization method is the z-score normalization, the normalization information acquiring section 41 acquires an average and a variance of a distribution of feature amounts as normalization coefficients.

When there is a gap between a response range for an emotion state for a reference model and a response range for the emotion state that is a range of a response expected for an application, the normalization coefficient converter 42 converts a normalization coefficient supplied by the normalization information acquiring section 41.

Specifically, the normalization information acquiring section 41 selects a reference model (for example, the reference model 61-1) supported by an application. Such a selection is performed in order to perform mapping (conversion) accurately.

On the basis of an input feature amount input by the normalization information acquiring section 41 and a feature amount used upon creation of a reference model selected by the normalization information acquiring section 41, the normalization coefficient converter 42 converts a normalization coefficient for the reference model. In other words, for example, the normalization coefficient converter 42 derives a conversion table g2( ) used to map a feature amount used upon creation to an input feature amount, where the feature amount used upon creation is used upon creation of the selected reference model 61-1, and the input feature amount is input by the normalization information acquiring section 41.

Note that, for example, the conversion table g2( ) may be derived in advance upon, for example, initial execution of an application, and may be stored in the normalization coefficient converter 42. In this case, the normalization coefficient converter 42 selects the stored conversion table g2( ).

The normalization coefficient converter 42 converts a normalization coefficient g1( ) using the derived conversion table g2( ) and outputs, to the section 25 for correcting a response range for a behavior state, a normalization coefficient g2(g1( )) obtained by the conversion.

Note that the normalization coefficient converter 42 may derive a conversion table for each reference model 61, or may derive a conversion table for one reference model 61 and obtain a conversion table for another reference model 61 using a correlation between feature amounts used upon creation of the respective reference models 61.

The section 25 for correcting a response range for a behavior state corrects a normalization coefficient in consideration of an impact of a state of a behavior of a user. In other words, for example, the section 25 for correcting a response range for a behavior state selects an adjustment gain table g3( ) corresponding to a behavioral context obtained from an IMU, and performs gain adjustment on the normalization coefficient obtained by the conversion being performed by the APP standard acquiring section 24.

The section 25 for correcting a response range for a behavior state outputs, to the normalization section 26, a normalization coefficient g3(g2(g1( ))) obtained by the gain adjustment.

The normalization section 26 includes a plurality of normalization sections 51-1 to 51-3 each provided for a corresponding one of reference models (for example, the reference models 61-1 to 61-3) included in the section 27 for chronologically labeling emotion states.

The normalization section 51-1 is provided correspondingly to the reference model 61-1. The normalization section 51-2 is provided correspondingly to the reference model 61-2. The normalization section 51-3 is provided correspondingly to the reference model 61-3. Note that the normalization sections 51-1 to 51-3 are referred to as the normalization sections 51 when there is no particular need to distinguish between the normalization sections 51-1 to 51-3.

The normalization section 51 performs a specified normalization on a feature amount of data supplied by the feature amount extracting section 23.

Specifically, the normalization section 51 normalizes a feature amount x of data supplied by the feature amount extracting section 23, using the normalization coefficient g3(g2(g1( )) supplied by the section 25 for correcting a response range for a behavior state, and outputs data g3(g2(g1(x)) obtained by the normalization to a corresponding reference model 61.

The section 27 for chronologically labeling emotion states includes the reference models 61-1 to 61-3, as described above.

When the data obtained by the normalization is input to the reference model 61 by a corresponding normalization section 51, the reference model 61 outputs a prediction label of an emotion state, the prediction label corresponding to a feature amount of the input data.

The stabilization processing section 28 includes stabilization processing sections 71-1 to 71-3 that respectively correspond to the reference models 61-1 to 61-3. Note that the stabilization processing sections 71-1-1 to 71-3 are referred to as stabilizations processing sections 71 when there is no particular need to distinguish between the stabilization processing sections 71-1-1 to 71-3.

Using chronological data of prediction labels of emotion states that is supplied by a corresponding reference model 61, the stabilization processing section 71 performs a weighted summation of the chronological prediction labels of the emotion states with the degrees of reliability of the prediction labels of the emotion states in a sliding window, and outputs a representative value of the prediction labels and a degree of reliability of the representative value of the prediction labels to the determination section 29 as an emotion estimation result.

The determination section 29 determines a state of an emotion of a target living body on the basis of the emotion estimation results supplied by the stabilization processing sections 71-1 to 71-3.

When a determination-target emotion state is a degree of arousal, the determination section 29 determines the degree of arousal of a target living body by a majority decision based on emotion estimation results (for example, degree-of-arousal information A, degree-of-arousal information B, and degree-of-arousal information C). It is assumed that, for example, the degree-of-arousal information A is information that indicates a high degree of arousal, the degree-of-arousal information B is information that indicates a low degree of arousal, and the degree-of-arousal information C is information that indicates a low degree of arousal. In this case, there are two votes for the low degree of arousal and one vote for the high degree of arousal, and thus the determination section 29 generates, as a result of a majority decision, a determination result showing that the degree of arousal is low.

The determination section 29 may determine a state of an emotion of a target living body using a method other than a majority decision. Further, the determination section 29 may estimate an emotion of a target living body only using a determination result corresponding to a selected reference model.

### <First Example of Adjusting Response Range>

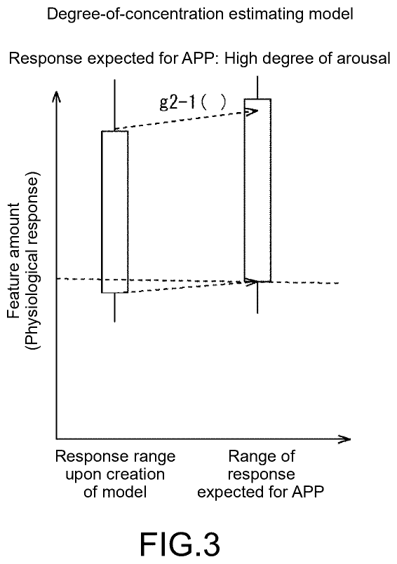

FIG. 3 illustrates a first example of adjusting a response range for an application.

In FIG. 3, a vertical axis represents a feature amount of a physiological response. A baseline that is indicated using a dashed line indicates that a biological state of a person is neutral. The feature amount is changed in an arousal direction (in a direction with a value larger than a value exhibited by a direction of the dashed line) when a person is more concentrated than in a neutral state, and the feature amount is changed in a relaxing direction (in a direction with a value smaller than a value exhibited by the direction of the dashed line) when the person is more relaxed than in the neutral state. The same applies to the figures described below.

For example, FIG. 3 illustrates the case in which an application used to estimate a degree of concentration and a degree-of-concentration estimating model corresponding to the application are used and in which a high degree of arousal is exhibited as a response expected for the application, where a response range (on the left in the figure) for a feature amount used upon creation of the model, is compared with a response range (on the right in the figure) for an input feature amount, the response range for an input feature amount being a range of a response expected when the application is used. Note that the degree-of-concentration estimating model corresponds to the reference model 61-3 described above.

Since the application used to estimate a degree of concentration is used, a value larger than a value of the baseline is exhibited overall in each of the response range for a feature amount used upon creation and the response range for an input feature amount, the response range for an input feature amount being a range of a response expected when an actual application is used.

Further, the response range for an input feature amount is larger than the response range for a feature amount used upon creation of a model, the response range for an input feature amount being a range of a response expected when an actual application is used.

In order to adjust this difference, the reference model 61-3 (a degree-of-concentration estimating model) based on type information that is obtained by acquiring an application trigger issued by an application when being started, is selected, and a conversion table g2-1( ) is derived.

### <Second Example of Adjusting Response Range>

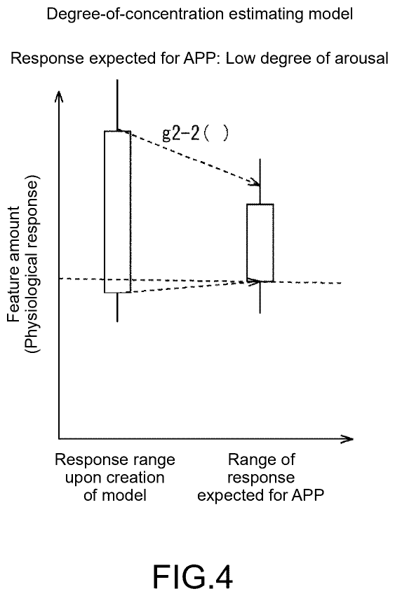

FIG. 4 illustrates a second example of adjusting a response range according to an application standard.

FIG. 4 illustrates the case in which an application used to estimate a degree of concentration and a degree-of-concentration estimating model corresponding to the application are used and in which a low degree of arousal is exhibited as a response expected for the application, where a response range (on the left in the figure) for a feature amount used upon creation of the model, is compared with a response range (on the right in the figure) for an input feature amount, the response range for an input feature amount being a range of a response expected when the application is used.

Since the application used to estimate a degree of concentration is used, a value larger than a value of the baseline is exhibited overall in each of the response range for a feature amount used upon creation of a model and the response range for an input feature amount, the response range for an input feature amount being a range of a response expected when an actual application is used.

Further, the response range for an input feature amount is considerably smaller than the response range for a feature amount used upon creation of a model, the response range for an input feature amount being a range of a response expected when an actual application is used.

In order to adjust this difference, the reference model 61-3 (a degree-of-concentration estimating model) based on type information that is obtained by acquiring an application trigger issued by an application when being started, is selected, and a conversion table g2-2( ) is derived.

### <Third Example of Adjusting Response Range>

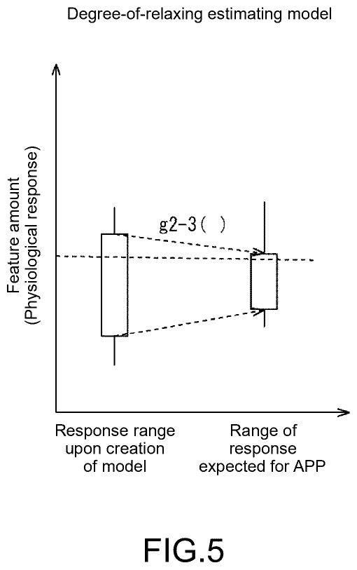

FIG. 5 illustrates a third example of adjusting a response range according to an application standard.

FIG. 5 illustrates the case in which an application used to estimate a degree of relaxing and a degree-of-relaxing estimating model corresponding to the application are used, where a response range (on the left in the figure) for a feature amount used upon creation of the model, is compared with a response range (on the right in the figure) for an input feature amount, the response range for an input feature amount being a range of a response expected when the application is used. Note that the degree-of-relaxing estimation corresponds to the reference model 61-1 described above.

Since the application used to estimate a degree of relaxing is used, a value smaller than a value of the baseline is exhibited overall in each of the response range for a feature amount used upon creation of a model and the response range for an input feature amount, the response range for an input feature amount being a range of a response expected when an actual application is used.

Further, the response range for an input feature amount is smaller than the response range for a feature amount used upon creation of a model, the response range for an input feature amount being a range of a response expected when an actual application is used.

In order to adjust this difference, the reference model 61-1 (a degree-of-relaxing estimating model) based on type information that is obtained by acquiring an application trigger issued by an application when being started, is selected, and a conversion table g2-3( ) is derived.

As described above, the emotion estimation processing apparatus 1 holds a plurality of reference models in advance for respective targets (standards) for started applications, where examples of the target include high and low degrees of arousal and a relaxing state. The conversion tables g2-1 to g2-3 are derived to be selected for the respective reference models. Then, an input feature amount is normalized using the selected conversion table.

<Example of Correcting Range According to Behavior State>

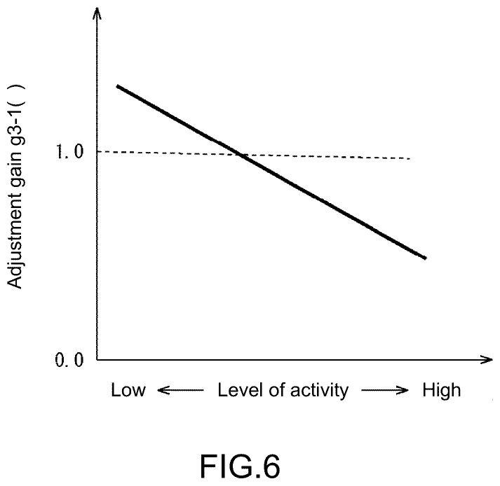

FIG. 6 illustrates an example of processing of correcting a range according to a behavior state.

In FIG. 6, a vertical axis represents a feature-amount gain. A response range for a feature amount is larger with a greater feature-amount gain, and the response range for a feature amount is smaller with a lesser feature-amount gain. A horizontal axis represents a level of activity.

A feature-amount gain table g3-1( ) exhibits a larger value of 1.0 or greater for a lower level of activity, and exhibits a value gradually made smaller for a higher level of activity. In other words, the feature-amount gain table g3-1( ) exhibits a monotonous reduction with respect to the activity level.

For example, an emotional physiological response corresponding to a state of a behavior of a person has characteristics in that a response sensitivity of a physiological response is decreased as the level of an activity state becomes higher (the level of activity becomes higher), as disclosed in, for example, Non-Patent Literature 1.

Thus, on the assumption of the characteristics in that a response sensitivity of a physiological response is decreased as a level of activity becomes higher, the section 25 for correcting a response range for a behavior state performs processing of making a response range for a feature amount smaller as the level of activity is increased, using the feature-amount gain table g3-1( ) illustrated in FIG. 6. In other words, processing of increasing an estimation sensitivity in estimating an emotion is performed in order to follow a weaker physiological response corresponding to a higher level of activity based on a behavioral context.

Processing such as the above-described adjustment of a response range for an application or the above-described correction of a response range depending on a behavior state is performed. The present technology enables the emotion estimation processing apparatus 1 illustrated in FIG. 1 to control a change in sensitivity of a physiological response, the sensitivity change being caused due to a behavioral context. This results in being able to achieve, according to an application, an optimal degree of accuracy in estimating an emotion.

As described above, the accuracy in estimating an emotion can be improved.

### <Emotion Estimation Processing>

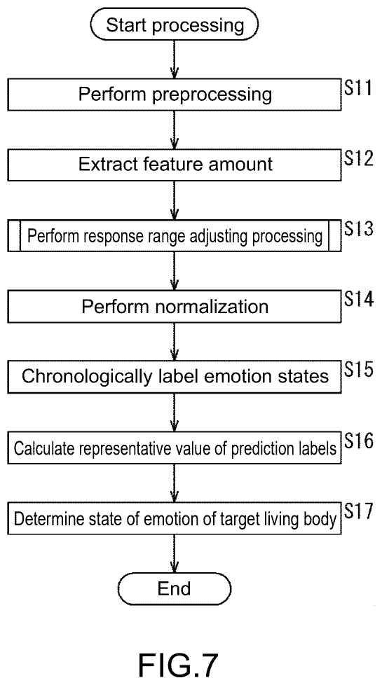

FIG. 7 is a flowchart used to describe emotion estimation processing performed by the emotion estimation processing apparatus 1.

In Step S11, the filter preprocessor 22 performs preprocessing such as bandpass filtering or denoising on a biological signal acquired by the sensor data acquiring section 21. The filter preprocessor 12 outputs, to the feature amount extracting section 23, the biological signal on which preprocessing has been performed.

In Step S12, the feature amount extracting section 23 extracts, using the biological signal supplied by the filter preprocessor 22, a feature amount as a model input variable used to estimate an emotion state. The feature amount extracting section 23 outputs the extracted feature amount to the APP standard acquiring section 24 and the normalization section 26.

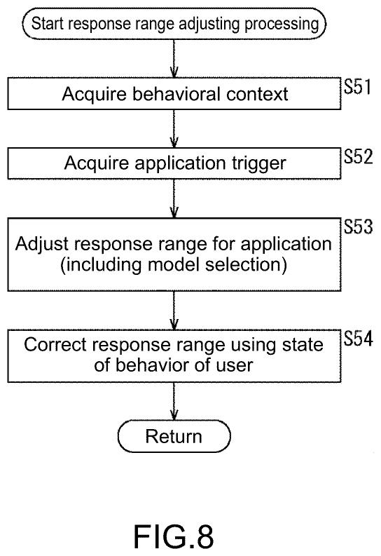

In Step S13, the APP standard acquiring section 24 and the section 25 for correcting a response range for a behavior state perform response range adjusting processing according to an application and a behavior state. This response range adjusting processing will be described later with reference to FIG. 8. In Step S13, the response range adjusting processing is performed according to an application, and response range correcting processing is performed according to a behavior state, as described with reference to FIGS. 3 to 6.

In Step S14, the normalization section 26 normalizes the feature amount supplied by the feature amount extracting section 23, using the normalization coefficient supplied by the section 25 for correcting a response range for a behavior state. The normalization section 26 outputs the normalized feature amount to the section 27 for chronologically labeling emotion states.

In Step S15, the section 27 for chronologically labeling emotion states chronologically labels emotion states. In other words, the section 27 for chronologically labeling emotion states uses, as input, chronological feature amounts, from among feature amounts supplied by the normalization section 26, that are in a sliding window. The section 27 for chronologically labeling emotion states performs identification on chronological prediction labels of emotion states using the reference models, and performs labeling.

The section 27 for chronologically labeling emotion states outputs, to the stabilization processing section 28, chronological data of prediction labels that is a result of chronologically labeling the emotion states. Here, degrees of reliability of the prediction labels that are obtained using the reference models are also output.

In Step S16, the stabilization processing section 28 calculates a representative value of the prediction labels. In other words, the stabilization processing section 28 calculates a degree of reliability of a representative value of the prediction labels in the sliding window using, as input, chronological emotion-state labels supplied by the section 27 for chronologically labeling emotion states. The stabilization processing section 28 performs threshold processing on a degree of reliability r of the representative value of the prediction labels using the formula (2) described above, and outputs a representative value z of the prediction labels as an emotion estimation result. The stabilization processing section 28 outputs, to the determination section 29 and as the emotion estimation result, the representative value of the prediction labels and the degree of reliability of the representative value of the prediction labels.

In Step S17, the determination section 29 determines a state of an emotion of a target living body using the representative value of the prediction labels and the degree of reliability of the representative value of the prediction labels, where the representative value and the degree of reliability are supplied by the stabilization processing section 28. The determination section 29 outputs, to an output side, a result of determining the state of the emotion of the target living body.

### <Response Range Adjusting Processing>

FIG. 8 is a flowchart used to describe the response range adjusting processing of Step S13 in FIG. 7.

In Step S51, the section 25 for correcting a response range for a behavior state acquires information related to a behavioral context supplied by an IMU. For example, the IMU acquires angular-velocity information and acceleration information, identifies a state of a behavior of a person, such as whether the person is staying quiet, walking, or taking exercise, on the basis of the acquired pieces of information, and identifies a behavioral context that is information indicating the identified behavior state. The IMU outputs information related to the identified behavioral context to the section 25 for correcting a response range for a behavior state. The section 25 for correcting a response range for a behavior state acquires the information related to the behavioral context, the information being output by the IMU.

In Step S52, the APP standard acquiring section 24 acquires an application trigger, and specifies a timing of the acquisition and the type of application. When, for example, an emotion meditation application is started, the started emotion meditation application issues an application trigger. The APP standard acquiring section 24 acquires the application trigger issued by the emotion meditation application, and specifies the emotion meditation application as the type of application.

In Step S53, the APP standard acquiring section 24 selects a reference model corresponding to the application of which the type has been specified, and adjusts a response range corresponding to a standard for the level of emotion state for the application of which the type has been specified.

In other words, the normalization coefficient converter 42 converts, according to a response range for an emotion state for an application, a normalization coefficient for a feature amount used upon creation of a selected reference model, on the basis of an input feature amount input by the normalization information acquiring section 41 and the feature amount used upon creation of the reference model, as described above.

Specifically, for example, the normalization coefficient converter 42 derives a conversion table g2( ) used to map, to an input feature amount, a feature amount used upon creation of a selected reference model, where the input feature amount is input by the normalization information acquiring section 41. The normalization coefficient converter 42 converts a normalization coefficient using the derived conversion table g2( ). A normalization coefficient g2(g1( )) obtained by the conversion is output to the section 25 for correcting a response range for a behavior state.

In Step S54, the section 25 for correcting a response range for a behavior state corrects a response range using a state of a behavior of a user.

In other words, for example, the section 25 for correcting a response range for a behavior state selects an adjustment gain table g3( ) that is used to perform gain adjustment and that corresponds to a behavioral context obtained from an IMU, and performs gain adjustment on the normalization coefficient g2(g1( )) obtained by the conversion being performed by the APP standard acquiring section 24.

The section 25 for correcting a response range for a behavior state outputs, to the normalization section 26, a normalization coefficient g3(g2(g1( ))) obtained by performing gain adjustment.

In addition to consideration of a standard for the level of emotion state depending on an application, an impact that a behavioral context has on an emotional physiological response is further considered in an operation environment of an actual application, as described above. This makes it possible to provide an emotion estimation algorithm in further consideration of a standard for the level of emotion for each application. This makes it possible to improve the accuracy in estimating an emotion in real time, and thus to expect an increase in a variety of application types.

### 2. Second Embodiment (Addition of Signal Quality Determining Section)

### <Example of Configuration of Emotion Estimation Processing Apparatus>

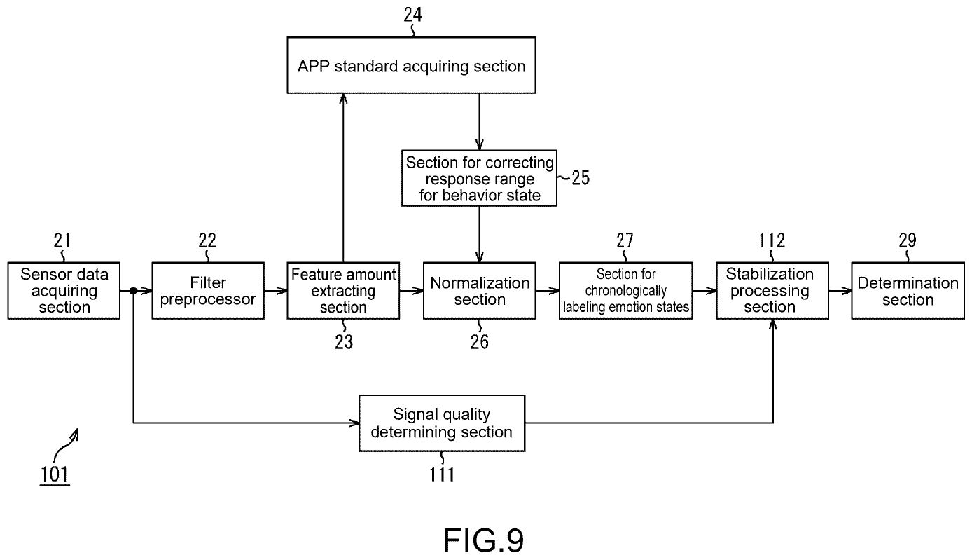

FIG. 9 is a block diagram of an example of a configuration of an emotion estimation processing apparatus according to a second embodiment of the present technology.

A signal quality determining section 111 is added to an emotion estimation processing apparatus 101 illustrated in FIG. 9, in order to further improve robustness of emotion estimation against noise when the noise is produced in an actual environment due to, for example, body movement.

In other words, the emotion estimation processing apparatus 101 illustrated in FIG. 9 is different from the emotion estimation processing apparatus 1 illustrated in FIG. 1 in that the signal quality determining section 111 is added and the stabilization processing section 28 is replaced with a stabilization processing section 112. In FIG. 9, a structural element corresponding to that in FIG. 1 is denoted by the same reference numeral as FIG. 1.

The signal quality determining section 111 analyzes a waveform of a biological signal acquired by the sensor data acquiring section 21, and identifies the type of artifact (such as noise other than a target signal). Examples of the type of artifact include ocular movement noise, myoelectric potential noise, eyeblink noise, and electro-cardio potential noise. The signal quality determining section 111 determines a signal quality on the basis of a result of the identification, and calculates a signal quality score as a result of determining a signal quality.

The stabilization processing section 112 performs a weighted summation with degrees of reliability of prediction labels of emotion states and the signal quality score that is a result of the determination performed by the signal quality determining section 111, and outputs a representative value of the prediction labels and a degree of reliability of the representative value of the prediction labels as an emotion estimation result.

The signal quality determining section 111 calculates chronological data of the signal quality scores, and outputs the calculated data to the stabilization processing section 112. Using the signal quality scores, the stabilization processing section 112 calculates the degree of reliability of the representative value of the prediction labels, with a signal quality being fed back to the calculation as weight. A method for calculating a degree of reliability r of a representative value by feeding back a signal quality can be defined using a formula (3) indicated below on the basis of the formula (1) described above.

$$[ Math . 3 ]$$

$$r ( t ) = {\sum }_{i} {w}_{i} {c}_{i} {s}_{i} {y}_{i} Δ {t}_{i} / {\sum }_{i} {w}_{i} Δ {t}_{i} \qquad ( 3 )$$

Note that sᵢ represents a signal quality score [0.0 1.0] of an i-th event.

With respect to degrees of reliability of prediction labels of emotion states for a plurality of events detected in a sliding window, a degree of reliability of a representative value of prediction labels in a sliding window is calculated in the form of consecutive values of [−1 1] using the formula (3) described above.

Further, threshold processing is performed on a degree of reliability r that is output of the formula (3), and the degree of reliability is substituted for the formula (2) described above. Accordingly, a representative value z of prediction labels is calculated as an emotion estimation result.

Note that a formula (4) indicated below may be used instead of the formula (3) described above.

$$[ Math . 4 ]$$

$$r ( t ) = {\sum }_{i} {w}_{i} {c}_{i} {s}_{i} {y}_{i} Δ {t}_{i} / {\sum }_{i} {w}_{i} {s}_{i} Δ {t}_{i} \qquad ( 4 )$$

The formula (3) has characteristics in that a degree of reliability r is lower in a sliding window with a low signal quality. The formula (3) has such characteristics, whereas the formula (4) makes it possible to perform normalization due to its denominator having sᵢ. This also makes it possible to perform a unified emotion determination in sliding windows with different signal qualities.

The formula (4) can be used instead of the formula (3) when the formula (3) is used below.

Further, an existing signal quality determination may be used by the signal quality determining section 111, where a signal quality score (SQE score) specific to processing (the formula (3)) performed by the stabilization processing section 112 is further calculated on the basis of an existing technology used for the signal quality determination.

In the signal quality determining section 111, for example, an identification class used when each kind of noise is produced is defined in advance, and an identification model using supervised learning is created. With first letters of the respective words of “signal quality estimation” being used, an identification model used to determine the quality is hereinafter referred to as an SQE identification model, and an identification class defined in advance is hereinafter referred to as an SQE identification class.

The signal quality determining section 111 identifies the type of waveform using the SQE identification model. Then, the signal quality determining section 111 calculates a signal quality score s specific to the signal processing method defined using the formula (3) described above. The signal quality score s is calculated using a formula (5) indicated below.

$$[ Math . 5 ]$$

$${s}_{m} = {α}_{m} f ( {d}_{m} ) \qquad ( 5 )$$

Here, m represents an SQE identification class, αₘ represents a class label (a constant: [0,1], which is set in advance) that corresponds to the SQE identification class, dₘ represents a degree of reliability of a class label that is obtained using an SQE identification model ([0,1], which is dependent on an input signal), and f( ) represents a function, where f( ) is defined as an adjustment look-up table ([0,1], which is set in advance).

x is an adjustment term obtained by considering, according to the type of noise identified using an SQE identification class, a difference in performance of denoising performed by the filter preprocessor 22.

Further, when a brain-wave signal is determined to be clean in the signal quality determining section 111 using an SQE identification model, a weight is increased as a degree of reliability of a class label that is obtained using the SQE identification model becomes higher to cause the signal quality to be easily identified as a signal quality of a positive class in the formula (3). Thus, f( ) is a look-up table that exhibits a monotonous increase.

Here, the positive class is a class in which the signal quality is determined to be greater than a specified threshold. A negative class is a class in which the signal quality is determined to be less than the specified threshold and to have noise.

When a brain-wave signal is clean, the weight is set to exhibit a maximum, where α=1.0. When noise is produced in the brain-wave signal, the signal quality is caused not to be easily identified as a signal quality of a positive class as the degree of reliability of a class label that is obtained using the SQE identification model becomes higher in the formula (3). Thus, f( ) is a look-up table that exhibits a monotonous reduction.

α is adjusted according to an SQE identification class and a difference in performance of the filter preprocessor 22. For example, α is set to exhibit a relatively large value such that αₘ=0.9 when eyeblink noise is produced that is relatively easily removed using signal processing. α is set to exhibit a relatively small value such that αₘ=0.2 when, for example, myoelectric potential noise is produced that is difficult to be removed in principle using signal processing performed by the filter preprocessor 22.

Note that αₘ represents an adjustment term, and there are no restrictions on the value of αₘ.

f(dₘ) exhibits a monotonous increase when m represents a primary signal, and exhibits a monotonous reduction when m represents noise.

As described above, when the formula (5) described above is defined, a signal quality score s [0.0 1.0] is larger if a signal quality is higher, and the signal quality score s is smaller if the signal quality is lower. This results in the formula (5) being the signal processing method specific to the formula (3).

Further, the example of determining a signal quality at each time from all of channels using an SQE identification model has been described above. However, the case in which a signal quality is determined by the signal quality determining section 111 for each channel using an SQE identification model is also assumed.

### <Emotion Estimation Processing>

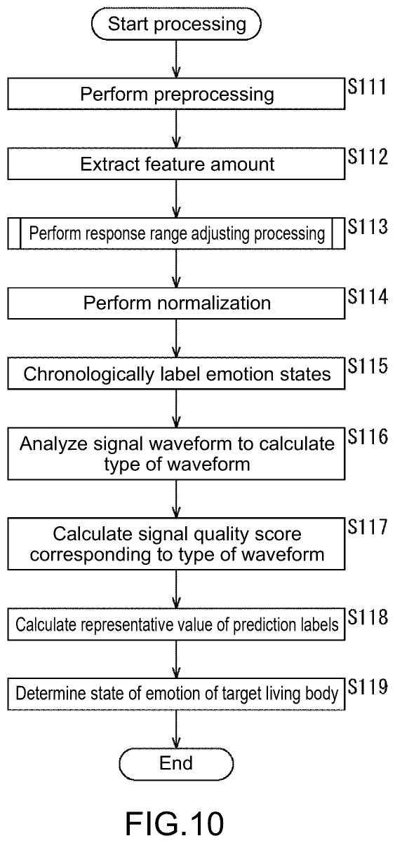

FIG. 10 is a flowchart used to describe emotion estimation processing performed by the emotion estimation processing apparatus 101 illustrated in FIG. 9.

The processes of Steps S111 to S115 in FIG. 10 are similar to the processes of Steps S11 to S15 in FIG. 7. Thus, descriptions thereof are omitted.

In FIG. 10, the processes of Steps S116 and S117 are performed in parallel with performing the processes of Steps S111 to S115.

In Step S116, the signal quality determining section 111 analyzes a signal waveform of a biological signal acquired by the sensor data acquiring section 21, and identifies the type of waveform.

In Step S117, the signal quality determining section 111 calculates a signal quality score corresponding to the type of waveform. The signal quality determining section 111 outputs the calculated signal quality score to the stabilization processing section 202.

In Step S118, the stabilization processing section 112 calculates a representative value of prediction labels. In other words, the stabilization processing section 112 calculates a degree of reliability r of a representative value of prediction labels in a sliding window by use of the formula (3) described above, using, as input, the chronological emotion-state labels supplied by the section 27 for chronologically labeling emotion states, and the signal quality score supplied by the signal quality determining section 111. The stabilization processing section 112 performs threshold processing on the degree of reliability r of the representative value of the prediction labels using the formula (2) described above, and outputs the representative value z of the prediction labels as an emotion estimation result.

In Step S119, the determination section 29 determines a state of an emotion of a target living body using the representative value of the prediction labels and the degree of reliability of the representative value of the prediction labels, where the representative value and the degree of reliability are supplied by the stabilization processing section 28. The determination section 29 outputs, to an output side, a result of determining the state of the emotion of the target living body.

As described above, an emotion estimation result is output on the basis of a result of performing a weighted summation with degrees of reliability of prediction labels and a result of determining a signal quality, and an emotion estimation result is output. Thus, the robustness is further improved with respect to an estimation accuracy in estimating an emotion, compared to the first embodiment.

Note that the example in which a signal quality is determined using an approach of machine learning has been described above. However, the determination may be performed using an approach other than machine learning. For example, the signal quality determining section 111 may output a signal quality score according to a degree of periodicity of the output signal, without using machine learning.

This technology can be applied to a biological signal with a high degree of periodicity, where examples of the biological signal include not only biological signals due to brain waves, mental sweating, and pulse waves but also biological signals due to, for example, a blood flow and a continuous blood pressure. Further, the present technology can also be applied to a biological signal due to, for example, breathing or an eyeblink.

### 3. Others

Note that the example in which a response range is corrected according to a context of a behavior state has been described above, although, for example, the context is not limited to the context of a behavior state. For example, a response range may be corrected according to a context of a location state.

For example, sensing is performed on a location context using the Global Navigation Satellite System (GNSS), and when it is determined that a user is in a green-space environment, a response range is corrected to perform multiplication by a gain that makes a range of a feature amount on the relaxing side larger.

### <Modifications of Range Correction>

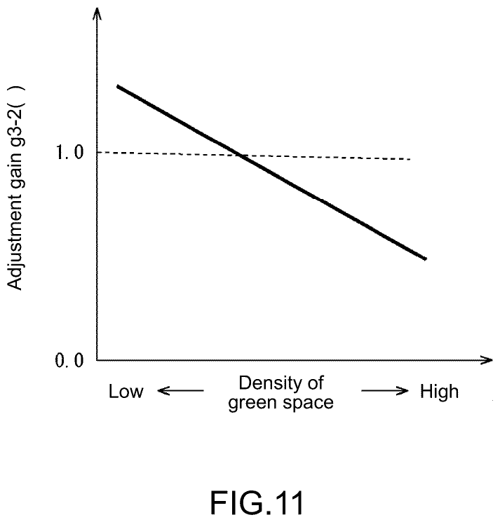

FIG. 11 illustrates an example of processing of correcting a range according to a location state.

In FIG. 11, a vertical axis represents a feature-amount gain. A range of a feature amount is larger with a greater feature-amount gain, and the range of a feature amount is smaller with a lesser feature-amount gain. A horizontal axis represents a degree of density of a green space.

A feature-amount gain table g3-2( ) exhibits a larger value of 1.0 or greater for a lower density of a green space, and exhibits a value gradually made smaller for a higher degree of density of the green space. In other words, the feature-amount gain table g3-2( ) exhibits a monotonous reduction with respect to the density of a green space.

It is known that, for example, a sensitivity of a physiological response that represents a relaxing state is increased when a user is in a green-space environment. In other words, conversely, a sensitivity of a physiological response that represents an arousal state is decreased when the user is in the green-space environment.

Therefore, in this case, characteristics in that a response sensitivity of a physiological response that represents an arousal state is decreased according to a location state (as a degree of density of a green space becomes higher) instead of a behavior state, are expected, and the feature-amount gain table g3-2( ) in FIG. 11 is used to perform processing of making a range for a feature amount smaller for a higher degree of density of a green space. In other words, processing of increasing an estimation sensitivity in estimating an emotion is performed in order to follow a weaker physiological response.

Moreover, the present technology can also be applied to processing that corresponds to, for example, a context of a thermal environment (whether the user is in a cold place or a warm place) or a context of a social environment (with whom a user is staying together).

### <Effects Provided by Present Technology>

In the present technology, a normalization coefficient is corrected according to a context related to a user (a certain state), and an input feature amount is normalized using the corrected normalization coefficient.

A change in sensitivity of a physiological response can be controlled, the sensitivity change being caused due to a behavioral context (a behavior state). Further, a reduction in the accuracy in estimating an emotion can be prevented and an experience of the user can be improved, the reduction being caused due to a reduction in sensitivity of an emotional physiological response corresponding to a state of a behavior of a user.

This makes it possible to improve the accuracy in estimating an emotion in real time.

Further, this makes it possible to expect an increase in a variety of actual application types that is caused along with a movement of a body of a user.

Deployment for various applications that is performed along with a body movement is expected, where examples of the various applications include applications to monitoring of a stressful state in everyday life, visualization of a state of being concentrated in an office environment, analysis on engagement of a user who is viewing moving-image content, and analysis on excitement during playing of a game.

### <Example of Configuration of Computer>

The series of processes described above can be performed using hardware or software. When the series of processes is performed using software, a program included in the software is installed from a program recording medium on, for example, a computer incorporated into dedicated hardware or a general-purpose personal computer.

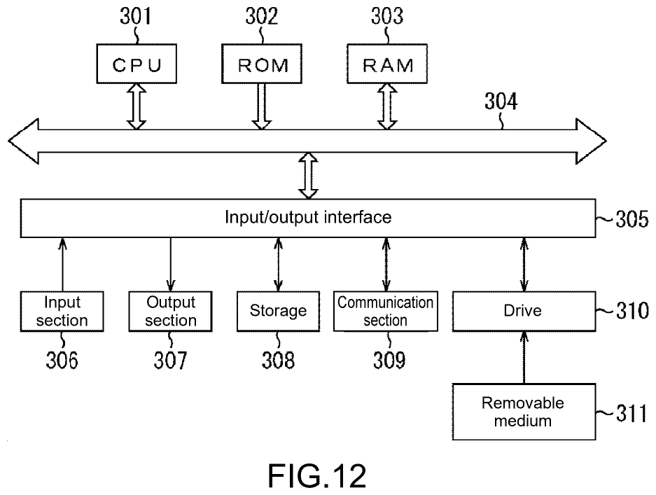

FIG. 12 is a block diagram of an example of a configuration of hardware of a computer that performs the series of processes described above using a program.

A central processing unit (CPU) 301, a read only memory (ROM) 302, and a random access memory (RAM) 303 are connected to each other through a bus 304.

Further, an input/output interface 305 is connected to the bus 304. An input section 306 that includes, for example, a keyboard and a mouse, and an output section 307 that includes, for example, a display and a speaker are connected to the input/output interface 305. Further, a storage 308 that includes, for example, a hard disk and a nonvolatile memory, a communication section 309 that includes, for example, a network interface, and a drive 310 that drives a removable medium 311 are connected to the input/output interface 305.

In a computer having the configuration described above, the series of processes described above is performed by the CPU 301 loading, for example, a program stored in the storage 308 into the RAM 303 and executing the program via the input/output interface 305 and the bus 304.

For example, the program executed by the CPU 301 is provided by being recorded in the removable medium 311 or provided via a wired or wireless transmission medium such as a local area network, the Internet, or digital broadcasting, and the provided program is installed on the storage 308.

Note that the program executed by the computer may be a program in which processes are chronologically performed in the order of the description herein, or may be a program in which processes are performed in parallel or a process is performed at a necessary timing such as a timing of calling.

Note that the system as used herein refers to a collection of a plurality of components (such as apparatuses and modules (parts)) and it does not matter whether all of the components are in a single housing. Thus, a plurality of apparatuses accommodated in separate housings and connected to one another via a network, and a single apparatus in which a plurality of modules is accommodated in a single housing are both systems.

Further, the effects described herein are not limitative but are merely illustrative, and other effects may be provided.

The embodiments of the present technology are not limited to the examples described above, and various modifications may be made thereto without departing from the scope of the present technology.

For example, the present technology may have a configuration of cloud computing in which a single function is shared to be cooperatively processed by a plurality of apparatuses via a network.

Further, the respective steps described using the flowcharts described above may be performed by a single apparatus, or may be shared to be performed by a plurality of apparatuses.

Furthermore, when a single step includes a plurality of processes, the plurality of processes included in the single step may be performed by a single apparatus, or may be shared to be performed by a plurality of apparatuses.

### <Example of Combination of Configurations>

The present technology may also take the following configurations.
(1) A signal processing apparatus, including:
   a feature amount extracting section that extracts an input feature amount on the basis of a measured biological signal;
   a response range correcting section that corrects a normalization coefficient according to a context related to a user;
   a normalization section that normalizes the input feature amount using the normalization coefficient corrected by the response range correcting section; and
   a section for chronologically labeling emotion states, the chronologically labeling section outputting a prediction label of an emotion state correspondingly to the normalized input feature amount using a machine learning model created in advance.
(2) The signal processing apparatus according to (1), in which the context is a behavioral context related to a behavior of the user.
(3) The signal processing apparatus according to (2), in which the response range correcting section corrects the normalization coefficient by performing multiplication by a monotonous-reduction gain according to an activity level based on the behavioral context.
(4) The signal processing apparatus according to any one of (1) to (3), further including
   an application adjustment section that adjusts, according to an application, a range for a feature amount used upon creation of the machine learning model, in which
   the response range correcting section corrects the normalization coefficient in the range for the feature amount used upon the creation, the range being adjusted by the application adjustment section.
(5) The signal processing apparatus according to (4), in which
   the application adjustment section
      selects the machine learning model according to the application,
      derives a conversion table used to convert, into a range for the input feature amount, the range for the feature amount used upon creation of the machine learning model, and
      adjusts the range for the feature amount used upon the creation by performing conversion on the normalization coefficient using the derived conversion table.
(6) The signal processing apparatus according to (4) or (5), further including:
   a stabilization processing section that outputs an emotion estimation result on the basis of a result of performing a weighted summation of the prediction labels using degrees of prediction label reliability that are degrees of reliability of the prediction labels; and
   a determination section that determines the emotion estimation result.
(7) The signal processing apparatus according to (6), further including
   a signal quality determining section that determines a signal quality of the biological signal, in which
   the stabilization processing section outputs the emotion estimation result on the basis of a result of performing a weighted summation of the prediction labels using the degrees of prediction label reliability and a result of determining the signal quality.
(8) The signal processing apparatus according to any one of (1) and (4) to (7), in which
   the context related to the user is a location context related to a location of the user.
(9) The signal processing apparatus according to any one of (1) to (8), in which
   the biological signal includes at least one of signals obtained by measuring a brain wave, mental sweating, a pulse wave, a blood flow, a continuous blood pressure, breathing, and an eyeblink.
(10) The signal processing apparatus according to any one of (1) to (9), further including
   a biological sensor that measures the biological signal.
(11) The signal processing apparatus according to any one of (1) to (10), in which
   a housing of the signal processing apparatus is wearable.
(12) A signal processing method, including:
   extracting an input feature amount on the basis of a measured biological signal;
   correcting a normalization coefficient according to a context related to a user;
   normalizing the input feature amount using the corrected normalization coefficient; and
   outputting a prediction label of an emotion state correspondingly to the normalized input feature amount using a machine learning model created in advance.

### REFERENCE SIGNS LIST

1 emotion estimation processing apparatus
21 sensor data acquiring section
22 filter preprocessor
23 feature amount extracting section
24 APP standard acquiring section
25 section for correcting response range for behavior state
26 normalization section
27 section for chronologically labeling emotion states
28 stabilization processing section
29 determination section
101 emotion estimation processing apparatus
111 signal quality determining section
112 stabilization processing section

1. A signal processing apparatus, comprising:
a feature amount extracting section that extracts an input feature amount on a basis of a measured biological signal;
a response range correcting section that corrects a normalization coefficient according to a context related to a user;
a normalization section that normalizes the input feature amount using the normalization coefficient corrected by the response range correcting section; and
a section for chronologically labeling emotion states, the chronologically labeling section outputting a prediction label of an emotion state correspondingly to the normalized input feature amount using a machine learning model created in advance.

2. The signal processing apparatus according to claim 1, wherein
the context is a behavioral context related to a behavior of the user.

3. The signal processing apparatus according to claim 2, wherein
the response range correcting section corrects the normalization coefficient by performing multiplication by a monotonous-reduction gain according to an activity level based on the behavioral context.

4. The signal processing apparatus according to claim 2, further comprising
an application adjustment section that adjusts, according to an application, a range for a feature amount used upon creation of the machine learning model, wherein
the response range correcting section corrects the normalization coefficient in the range for the feature amount used upon the creation, the range being adjusted by the application adjustment section.

5. The signal processing apparatus according to claim 4, wherein
the application adjustment section
selects the machine learning model according to the application,
derives a conversion table used to convert, into a range for the input feature amount, the range for the feature amount used upon creation of the machine learning model, and
adjusts the range for the feature amount used upon the creation by performing conversion on the normalization coefficient using the derived conversion table.

6. The signal processing apparatus according to claim 4, further comprising:
a stabilization processing section that outputs an emotion estimation result on a basis of a result of performing a weighted summation of the prediction labels using degrees of prediction label reliability that are degrees of reliability of the prediction labels; and
a determination section that determines the emotion estimation result.

7. The signal processing apparatus according to claim 6, further comprising
a signal quality determining section that determines a signal quality of the biological signal, wherein
the stabilization processing section outputs the emotion estimation result on a basis of a result of performing a weighted summation of the prediction labels using the degrees of prediction label reliability and a result of determining the signal quality.

8. The signal processing apparatus according to claim 1, wherein
the context related to the user is a location context related to a location of the user.

9. The signal processing apparatus according to claim 1, wherein
the biological signal includes at least one of signals obtained by measuring a brain wave, mental sweating, a pulse wave, a blood flow, a continuous blood pressure, breathing, and an eyeblink.

10. The signal processing apparatus according to claim 1, further comprising
a biological sensor that measures the biological signal.

11. The signal processing apparatus according to claim 1, wherein
a housing of the signal processing apparatus is wearable.

12. A signal processing method, comprising:
extracting an input feature amount on a basis of a measured biological signal;
correcting a normalization coefficient according to a context related to a user;
normalizing the input feature amount using the corrected normalization coefficient; and
outputting a prediction label of an emotion state correspondingly to the normalized input feature amount using a machine learning model created in advance.
